(19) United States
(12) Patent Application Publication
(10) Pub. No.: US 2015/0209171 A1
(43) Pub. Date: Jul. 30, 2015

(54) KNITTED FABRICS IN MEDICAL AIDS

(71) Applicant: BAUERFEIND AG, Zeulenroda-Triebes (DE)

(72) Inventor: Gerald Stier, Langenwetzendorf (DE)

(73) Assignee: BAUERFEIND AG, Zeulenroda-Triebes (DE)

(21) Appl. No.: 14/425,219

(22) PCT Filed: Aug. 16, 2013
(86) PCT No.: PCT/EP2013/067154

§ 371 Date: Mar. 2, 2015

(30) Foreign Application Priority Data
Sep. 7, 2012 (DE) 10 2012 017 722.4

(51) Int. Cl. A61F 5/02; D04B 21/20; D04B 21/18

(52) U.S. Cl. CPC A61F 5/02; D04B 21/18; D04B 21/20

(57) ABSTRACT

The invention relates to medical aids, e.g. bandages and orthoses, comprising at least one knitted fabric, in particular a warp-knitted fabric, especially warp-knitted fabric without loop pile, said fabric having an open structure that can be stretched simultaneously in the longitudinal and transverse directions. The invention also relates to the use of such knitted fabrics, especially warp-knitted fabrics, in medical aids, e.g. bandages and orthoses, and to methods for producing said medical aids.

The present invention relates to medical aids, for example bandages and orthoses, comprising at least one knitted fabric, in particular a warp-knitted fabric, especially a warp-knitted fabric without loop pile, said fabric having an open structure that can be stretched simultaneously in the longitudinal and transverse directions. The present invention also relates to the use of such knitted fabrics, especially warp-knitted fabrics, in medical aids, for example bandages and orthoses, and to methods for producing said medical aids.

Knitted fabrics include knitted fabrics and crocheted fabrics.

In the context of the present invention, the crocheted product, also called crocheted fabric, is understood as a knitted fabric produced by crocheting, that is, in particular not a braided fabric or woven fabric. The preferred crocheted fabric according to the invention can particularly be a crocheted compression fabric. Crocheted fabrics are produced by forming the stitches in succession using one thread or yarn.

In contrast, knitted fabrics, also called knits or knitted products, are fabrics that are manufactured industrially on a knitting machine by forming stitches using thread systems. They belong to the group of knitted fabrics. Differentiation is made between weft-knitted fabrics and warp-knitted fabrics. Whereas with crocheting, stitches are produced side by side, so that the thread runs horizontally along a row of stitches, in a knitted fabric, the threads form stitches one above the other, with the thread running vertically and forming a stitch connector with the adjacent thread. A warp-knitted fabric is produced using multiple threads, and at least the same number of needles. In warp-knitted fabrics, the threads run vertically and are picked up by the needles and pulled through the preceding row of stitches.

Warp-knitted fabric is used, for example, in the form of a mesh, for example, in the form of luggage mesh in automobiles. Such meshes can be designed as elastic or inelastic. However maintaining more precise parameters with regard to elasticity, for example mesh having a precisely defined force-stretch behavior, plays no role or at most a subordinate role in such cases.

The object of the invention is to provide medical aids that comprise novel and enhanced knitted fabrics. In particular, the knitted fabrics used in the medical aids are to have the desired force-stretch parameters, along with good breathability. The knitted fabric should also be elastic enough to adjust well to the shape of the body.

The object of the invention is further to provide new areas of application for knitted fabrics, in particular crocheted fabrics and/or knitted fabrics, preferably warp-knitted fabrics, in particular warp-knitted fabrics without loop pile in the form of a mesh.

The technical object of the invention is preferably attained by providing a medical aid, uses and methods according to the main claim and the secondary claims, in particular by a medical aid comprising at least one mesh-like knitted fabric, said mesh-like knitted fabric having an open structure that can be stretched simultaneously in both the longitudinal and transverse directions, and is preferably used in the stretched state in the medical aid. Each of the sub-elements of the open structure preferably has a width of at least 2.0 mm to at most 10.0 mm in the stretched state. Alternatively or additionally, each of the sub-elements of the open structure preferably has a height of at least 2.0 mm to at most 10.0 mm in the stretched state.

According to the invention, the at least one knitted fabric, in particular the crocheted fabric and/or knitted fabric, preferably a warp-knitted fabric, is mesh-like, preferably embodied as a mesh. The knitted fabric, in particular the crocheted fabric and/or knitted fabric, preferably the warp-knitted fabric, is therefore preferably in the form of a mesh, i.e. it is an open-pored textile structure with stitches, designed as openings, at regular intervals. The stitches or openings can be designed as rhombic, square, hexagonal, diamond-shaped or honeycombed, for example. A mesh structure preferably consists of a plurality of the same or at least similar sub-structures. In the context of the present invention, a sub-structure is understood as a stitch or an opening and the threads that surround and delimit the stitch or opening.

In the context of the present invention, an open structure of a knitted fabric, in particular a crocheted fabric and/or knitted fabric, preferably a warp-knitted fabric, is understood in particular as a mesh-like structure that has a plurality of openings and stitches, in particular larger openings, preferably openings having a width and/or height of at least 2.0 mm, more preferably least 3.0 mm in the stretched state. An open structure is therefore particularly an open-pored mesh.

In the text of the present application, all specifications relating to the height and/or width of the openings, sub-elements and/or stitches relate to the height and/or width of the openings, sub-elements and/or stitches in the employed and utilized state, that is, in the stretched state, of the knitted fabric in the medical aid, unless otherwise indicated.

In the context of the present invention, the phrase “in the stretched state” is understood as the state in which the knitted fabric is used in the medical aid. According to the invention, the knitted fabric is preferably used in the medical aid not in the unstretched state, but preferably in a pre-stretched state. The knitted fabric in the medical aid is preferably not stretched to its maximum, and can be stretched further as the medical aid is applied to the body of a human or animal.

In a preferred embodiment of the present invention, the knitted fabric is a crocheted fabric, in particular a circular- or flat-crocheted fabric. In a preferred embodiment, the knitted fabric is a flat-crocheted fabric. In a preferred embodiment, the knitted fabric is a circular-crocheted fabric. In a preferred embodiment, the knitted fabric is three-dimensional. In a preferred embodiment, the knitted fabric is a three-dimensional flat-crocheted fabric, for example a bandage. In a preferred embodiment of the present invention, the crocheted fabric has weft threads, also referred to as inlays and/or inlay threads.

In a preferred embodiment of the present invention, the knitted fabric is a knitted fabric, in particular a warp-knitted fabric.

In a preferred embodiment, the knitted fabric has no pile threads.

In a further embodiment of the present invention, a medical aid comprising a particularly mesh-like knitted fabric is provided, wherein said knitted fabric is a knitted fabric, in particular a warp-knitted fabric, which is characterized in that the knitted fabric has no pile threads and has an open structure that can be stretched simultaneously in the longitudinal and transverse directions.

In a preferred embodiment, the knitted fabric has a ground thread and at least one additional thread, in particular a weft thread.

In a preferred embodiment, the thickness of the ground thread is greater than the thickness of the at least one additional thread, in particular the weft thread.

In another preferred embodiment, the thickness of the ground thread is less than the thickness of the at least one additional thread, in particular the weft thread.

In a preferred embodiment of the invention, the knitted fabric, preferably the warp-knitted fabric, has no pile threads, and is thus without loop pile. Pile threads are threads that are looped, which can produce three-dimensionality, for example.

The inventors of the present invention unexpectedly found that a knitted fabric, in particular a warp-knitted fabric, in particular a knitted fabric without loop pile, in particular a warp-knitted fabric without loop pile, can be used as a knitted fabric in medical aids, particularly if said fabric has an open structure. It was unexpectedly found that the knitted fabric, in particular the warp-knitted fabric, is an especially good substitute for conventional knitted fabrics.

The inventors of the present invention found not only that the knitted fabric, in particular the crocheted fabric and/or knitted fabric, preferably the warp-knitted fabric, can be designed such that it can be stretched in the longitudinal and transverse directions, in particular simultaneously stretched in both directions, but also that the knitted fabric, in particular the crocheted fabric and/or knitted fabric, preferably the warp-knitted fabric, can be designed such that it meets to the required force-stretch specifications with a high degree of precision, and can therefore have a desired and specific amount of stretch when a specific amount of force is applied. Furthermore, the knitted fabric, in particular the crocheted fabric and/or knitted fabric, preferably the warp-knitted fabric, is able to breathe through the stitches and openings, which preferably have a width and/or height of at least 2 mm and at most 10 mm in the stretched state. It has been found that a knitted fabric, in particular a crocheted fabric and/or knitted fabric, preferably a warp-knitted fabric, has substantially better breathability than knitted fabrics previously used in the prior art. As a result, heat is prevented from accumulating, thereby reducing perspiration. The knitted fabric used according to the invention, in particular the crocheted fabric and/or knitted fabric, preferably the warp-knitted fabric, has an elasticity that allows it to adjust well to the shape of the body, with the two-dimensional surface adapting optimally to the three-dimensional body of the patient. This can be achieved particularly by designing the openings as rhombic. The inventors also found that using a knitted fabric, in particular a crocheted fabric and/or knitted fabric, preferably a warp-knitted fabric, can lead to reduced wrinkling.

The use according to the invention of a knitted fabric, in particular a crocheted fabric and/or knitted fabric, preferably a warp-knitted fabric, in particular a warp-knitted fabric as defined above, in a medical aid, such as a bandage or an orthosis, thus leads to improved breathability with a simultaneously precisely predefined compressive effect.

The special design of the knitted fabric, in particular the crocheted fabric and/or knitted fabric, preferably the warp-knitted fabric, allows new design effects, in particular structural effects, to advantageously be generated, since longitudinal strips in different colors of yarn are possible.

Such a knitted fabric, in particular a crocheted fabric and/or knitted fabric, preferably a warp-knitted fabric, can advantageously be stretched in both the transverse direction and the longitudinal direction.

In a preferred embodiment, the knitted fabric, preferably the crocheted fabric and/or the knitted fabric, preferably the warp-knitted fabric, is stretched in at least one direction in the medical aid. The knitted fabric, preferably the crocheted fabric and/or knitted fabric, preferably the warp-knitted fabric is thus preferably used in the medical aid, for example welded or bonded thereto, such that when the medical aid is in the unapplied and unstretched form, the fabric is stretched, preferably pre-stretched, in at least one direction.

A person skilled in the art could easily determine the corresponding parameters of the knitted fabric, preferably the crocheted fabric and/or knitted fabric, preferably the warp-knitted fabric, and could produce a knitted fabric, preferably a crocheted fabric and/or a knitted fabric, preferably a warp-knitted fabric having these parameters.

In a preferred embodiment, the medical aid is an orthopedic aid. In a preferred embodiment, the medical aid is a bandage or an orthosis. In a preferred embodiment, the medical aid is a bandage. In an alternative embodiment, the medical aid is an orthosis.

In a preferred embodiment, the medical aid is a back brace. In an alternative embodiment, the medical aid is an arm bandage or foot bandage.

In a preferred embodiment, the knitted fabric, preferably the crocheted fabric and/or knitted fabric, preferably the warp-knitted fabric is embodied as a base support for a bandage.

In a preferred embodiment, the open structure of the knitted fabric, preferably the crocheted fabric and/or knitted fabric, preferably the warp-knitted fabric, is a mesh structure, a rhombic structure or a honeycomb structure. In a preferred embodiment, the open structure of the knitted fabric, preferably the crocheted fabric and/or knitted fabric, preferably the warp-knitted fabric, is a rhombic structure or a honeycomb structure. In a preferred embodiment, the open structure of the knitted fabric, preferably the crocheted fabric and/or knitted fabric, preferably the warp-knitted fabric, is a rhombic structure.

In a preferred embodiment, each of the individual sub-elements of the structure, in particular of the rhombic structure, has a width and/or height of at least 2 mm and at most 8 mm. In a preferred embodiment, each of the individual sub-elements of the structure, in particular of the rhombic structure, has a width and/or height of at least 2 mm, in particular of at least 3 mm. In a preferred embodiment, each of the individual sub-elements of the structure, in particular of the rhombic structure, has a width and/or height of at most 8 mm, in particular at most 6 mm. In a preferred embodiment, each of the individual sub-elements of the structure, in particular of the rhombic structure, has a width and a height of at least 2 mm and at most 8 mm, in particular at most 6 mm. In a preferred embodiment, each of the individual sub-elements of the structure, in particular of the rhombic structure, has a width of at least 2 mm and at most 6 mm. In a preferred embodiment, each of the individual sub-elements of the structure, in particular of the rhombic structure, has a height of at least 2 mm and at most 6 mm.

In a preferred embodiment, when the medical aid is in the unapplied and unstretched state, each of the individual sub-elements of the structure, in particular of the rhombic structure, has a width and/or height of at least 0.5 mm and at most 5 mm, preferably at least 0.7 mm and at most 4 mm, preferably at least 0.9 mm and at most 3 mm, preferably at least 1 mm and at most 3 mm in the unstretched state. In a preferred embodiment, when the medical aid is in the applied and stretched state, each of the individual sub-elements of the structure, in particular of the rhombic structure, has a width and/or height of at least 2 mm and at most 10 mm.

In a preferred embodiment, in the stretched state, preferably in the maximum stretched state, each of the sub-elements of the structure, in particular of the rhombic structure or honeycomb structure, has a width and/or height of at least 2 mm, preferably at least 2 and at most 10 mm, preferably at least 3 and at most 8 mm, preferably at least 3 and at most 7 mm, preferably at least 3 and at most 6 mm, preferably at least 5 and at most 6 mm.

In a preferred embodiment, the sub-elements have the same elongation in the longitudinal direction and the transverse direction. In the stretched state, the height and width of the sub-elements of the structure present in the knitted fabric are preferably approximately equal, preferably equal.

In a preferred embodiment, in the unapplied and unstretched form, each of the individual sub-elements of the structure, in particular of the rhombic structure, has a width and/or height of at least 0.5 mm, and in the applied and stretched form in the medical aid each of said sub-elements has a width and/or height of at most 8 mm.

Sub-elements of the structure, in particular of the rhombic structure, having the measurements specified here, advantageously lead to a reduction or even a prevention of edema formation.

In a preferred embodiment, the knitted fabric, preferably the crocheted fabric and/or knitted fabric, preferably the warp-knitted fabric, contains at least one rubbery-elastic thread.

In a preferred embodiment, the knitted fabric, preferably the crocheted fabric and/or knitted fabric, preferably the warp-knitted fabric, contains at least one rubbery-elastic thread and at least one wrapped yarn.

In a preferred embodiment, the knitted fabric, preferably the crocheted fabric and/or knitted fabric, preferably the warp-knitted fabric, consists of at least one rubbery-elastic thread and at least one wrapped yarn. In a preferred embodiment, the knitted fabric, preferably the crocheted fabric and/or knitted fabric, preferably the warp-knitted fabric, consists of rubbery-elastic threads and wrapped yarn.

In a preferred embodiment, one component of the knitted fabric, preferably the crocheted fabric and/or knitted fabric, preferably the warp-knitted fabric, consists of a double-wrapped rubbery-elastic thread. In a preferred embodiment, in each case one sub-element of the knitted fabric, preferably the crocheted fabric and/or knitted fabric, preferably the warp-knitted fabric, consists of a double-wrapped, rubbery-elastic thread. In a preferred embodiment, the knitted fabric, preferably the crocheted fabric and/or knitted fabric, preferably the warp-knitted fabric, consists of a double-wrapped rubbery-elastic thread.

Wrapped rubber threads advantageously lead to good longitudinal elasticity of the knitted fabric, preferably the crocheted fabric and/or knitted fabric, preferably the warp-knitted fabric.

In a preferred embodiment, the rubbery-elastic thread is made of a thermoplastic thread material.

In a preferred embodiment, the knitted fabric, preferably the crocheted fabric and/or knitted fabric, preferably the warp-knitted fabric, comprises a thread, preferably a warp thread having a gauge or thickness of at least 700 dtex. In a preferred embodiment, the knitted fabric, preferably the crocheted fabric and/or knitted fabric, preferably the warp-knitted fabric, comprises a thread, preferably a warp thread, having a gauge or thickness of at most 10.5 ktex. In a preferred embodiment, the knitted fabric, preferably the crocheted fabric and/or knitted fabric, preferably the warp-knitted fabric, comprises a warp thread having a gauge or thickness of at least 700 dtex to at most 10.5 ktex. In a preferred embodiment, the knitted fabric, preferably the crocheted fabric and/or knitted fabric, preferably the warp-knitted fabric, comprises a thread, preferably a warp thread having a gauge or thickness of at least 850 dtex to at most 1050 dtex. In a preferred embodiment, the knitted fabric, preferably the crocheted fabric and/or knitted fabric, preferably the warp-knitted fabric, comprises a thread, preferably a warp thread having a gauge or thickness of at least 900 dtex to at most 999 dtex. In a preferred embodiment, the knitted fabric, preferably the crocheted fabric and/or knitted fabric, preferably the warp-knitted fabric, comprises a thread, preferably a warp thread, having a gauge or thickness of at least 850 dtex. In a preferred embodiment, the knitted fabric, preferably the crocheted fabric and/or knitted fabric, preferably the warp-knitted fabric, comprises a thread, preferably a warp thread having a gauge or thickness of approximately 940 dtex.

It was found that the force-stretch behavior of the warp-knitted fabric could be precisely defined and adjusted based on the choice of the material and/or the sub-elements of the knitted fabric, preferably the crocheted fabric and/or knitted fabric, preferably the warp-knitted fabric.

In a preferred embodiment, the knitted fabric, preferably the crocheted fabric and/or knitted fabric, preferably the warp-knitted fabric, has approximately 30% stretch with an applied force of 1.0 to 5.0 N/cm, preferably approximately 2.5 N/cm. In a particularly preferred embodiment, the knitted fabric, preferably the crocheted fabric and/or knitted fabric, preferably the warp-knitted fabric, has 30% stretch with an applied force of 1.0 to 5.0 N/cm, preferably 2.5 N/cm.

In a preferred embodiment, in the medical aid the knitted fabric, preferably the crocheted fabric and/or knitted fabric, preferably the warp-knitted fabric, is stretched in at least one direction, preferably in the longitudinal direction and/or the transverse direction, by at least 10% and at most 90%, preferably at least 15% and at most 80%, preferably at least 15% and at most 70%, preferably at least 20% and at most 50%, preferably at least 20% and at most 40%, as compared with the unstretched state of the knitted product.

In a preferred embodiment, the knitted fabric, preferably the crocheted fabric and/or knitted fabric, preferably the warp-knitted fabric, serves as a compression element in the medical aid.

In a preferred embodiment, at least one shaping element, one support element and/or one zone of inelastic material is fastened onto the knitted fabric, preferably the crocheted fabric and/or the knitted fabric, preferably the warp-knitted fabric. In a preferred embodiment, at least one shaping element, one support element or one zone of inelastic material is fastened onto the knitted fabric, preferably the crocheted fabric and/or knitted fabric, preferably the warp-knitted fabric.

In a preferred embodiment, the at least one shaping element, support element or at least one inelastic element is welded, stitched, attached by hook-and-loop tape, riveted or bonded to the knitted fabric, preferably the crocheted fabric and/or knitted fabric, preferably the warp-knitted fabric. In a preferred embodiment, the at least one shaping element, support element or at least one inelastic element is welded or bonded to the knitted fabric, preferably the crocheted fabric and/or knitted fabric, preferably the warp-knitted fabric. In a preferred embodiment, the at least one shaping element, support element or at least one inelastic element is welded to the knitted fabric, preferably the crocheted fabric and/or knitted fabric, preferably the warp-knitted fabric. It was found that the knitted fabric, preferably the crocheted fabric and/or knitted fabric, preferably the warp-knitted fabric disclosed herein can be readily thermally processed, for example by means of ultrasonic cutting and/or heat cutting and/or by means of ultrasonic welding and/or high-frequency welding, when a thermoplastic thread material is used.

The invention also attains the stated technical object by using a knitted fabric, preferably a crocheted fabric and/or knitted fabric, preferably a warp-knitted fabric, in particular a knitted fabric, preferably a crocheted fabric and/or knitted fabric, preferably a warp-knitted fabric according to the invention, or a knitted fabric, preferably a crocheted fabric and/or knitted fabric, preferably a warp-knitted fabric as described above in a medical aid.

In a preferred embodiment, the knitted fabric, preferably the crocheted fabric and/or knitted fabric, preferably the warp-knitted fabric is used in a bandage or orthosis. In a preferred embodiment, the knitted fabric, preferably the crocheted fabric and/or knitted fabric, preferably the warp-knitted fabric, is used in a bandage. In a preferred embodiment, the knitted fabric, preferably the crocheted fabric and/or knitted fabric, preferably the warp-knitted fabric, is used in a bandage as a base support for a bandage.

In a preferred embodiment, the knitted fabric, preferably the crocheted fabric and/or knitted fabric, preferably the warp-knitted fabric, is used as a compression element in the medical aid.

The present invention also relates to methods for producing a medical aid, said method comprising the following steps: a) prestressing a knitted fabric, preferably a crocheted fabric and/or knitted fabric, preferably a warp-knitted fabric; and b) applying the at least one shaping element, support element and/or inelastic element to the prestressed knitted fabric, preferably crocheted fabric and/or knitted fabric, preferably warp-knitted fabric.

In a preferred embodiment, the medical aid is a medical aid according to the invention. In a preferred embodiment, the knitted fabric, preferably the crocheted fabric and/or knitted fabric, preferably the warp-knitted fabric is a knitted fabric, preferably a crocheted fabric and/or knitted fabric, preferably a warp-knitted fabric according to the invention, or a knitted fabric, preferably a crocheted fabric and/or knitted fabric, preferably a warp-knitted fabric as described above.

In a preferred embodiment, in step b) the at least one shaping element, support element and/or inelastic element is welded, stitched, attached by hook-and-loop tape, riveted or bonded to the prestressed knitted fabric, preferably crocheted fabric and/or knitted fabric, preferably warp-knitted fabric. In a preferred embodiment, in step b) the at least one shaping element, support element or inelastic element is welded, stitched, attached by hook-and-loop tape, riveted or bonded to the prestressed knitted fabric, preferably crocheted fabric and/or knitted fabric, preferably warp-knitted fabric. In a preferred embodiment, in step b) the at least one shaping element, support element or inelastic element is welded or bonded to the prestressed knitted fabric, preferably crocheted fabric and/or knitted fabric, preferably warp-knitted fabric. In a preferred embodiment, in step b) the at least one shaping element, support element or inelastic element is welded, for example via ultrasound welding methods, to the prestressed knitted fabric, preferably crocheted fabric and/or knitted fabric, preferably warp-knitted fabric.

Step a) can naturally also be preceded by additional steps, for example mechanical production of the knitted fabric, preferably the crocheted fabric and/or knitted fabric, preferably the warp-knitted fabric, and stamping or cutting the knitted fabric, preferably the crocheted fabric and/or knitted fabric, preferably the warp-knitted fabric to size, for example by heat cutting or laser cutting.

Preferred embodiments are specified in the dependent claims.

In the following, the invention will be specified in greater detail in reference to the embodiment examples represented in the set of drawings, without restricting the invention to these examples.

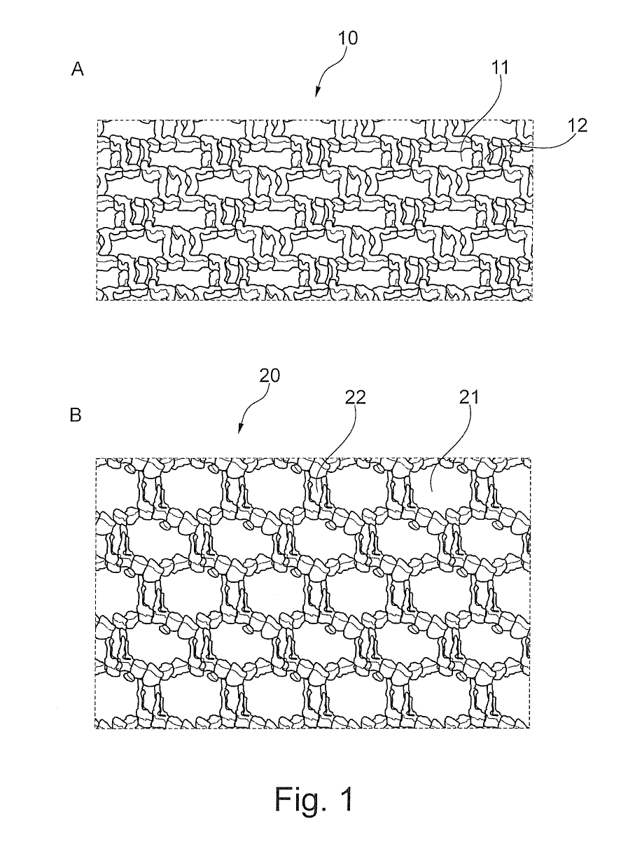

In the drawings, FIG. 1 shows a section of a warp-knitted fabric according to the invention with a plurality of sub-elements. In A, the warp-knitted fabric is shown in an unstretched state. In B, the warp-knitted fabric is shown in a stretched state.

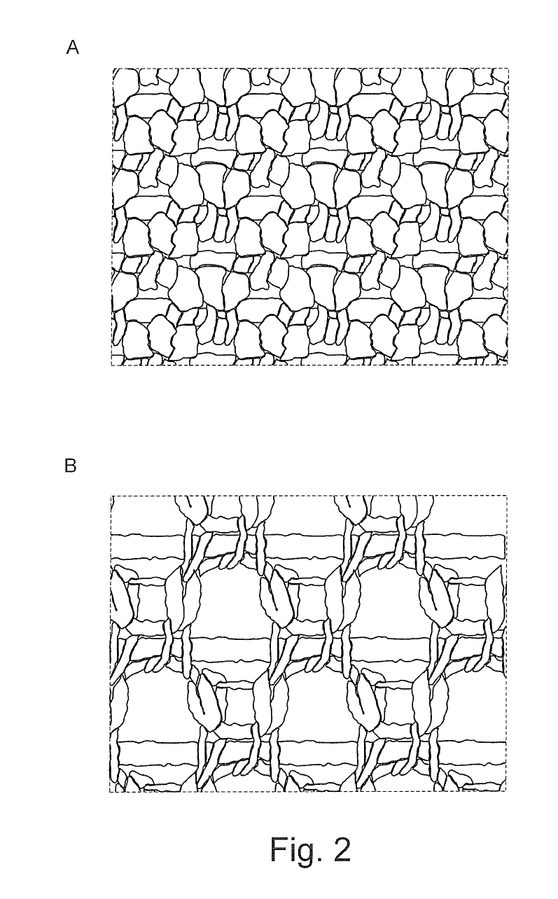

FIG. 2 shows a section of the crocheted fabric comprising a plurality of sub-elements, according to the invention. In A, the crocheted fabric is shown in an unstretched state. In B, the crocheted fabric is shown in a stretched state.

### EXAMPLE

A warp-knitted fabric comprising a double-wrapped raw rubber thread was produced by machine. The threads used to form the stitches had a titer of 940 dtex. The figure shows a section of this warp-knitted fabric (10, 20). The warp-knitted fabric (10, 20) is mesh-like. The warp-knitted fabric (10, 20) consists of a plurality of sub-elements, each sub-element consisting of an opening (11, 21) and the threads (12, 22) that surround the opening (11, 21), which threads also simultaneously delimit the opening (10, 20), and therefore determine the size of the opening. In A, the warp-knitted fabric (10) is not stretched. In B, the warp-knitted fabric (20) is stretched using a specific amount of force. Stretching the warp-knitted fabric (20) causes the structure (22) to open, so that the openings (21) enlarge in the direction of stretch.

The warp-knitted fabric can then be cut to the proper size, for example, and can function as the base support in a back brace. Additional elements of the back brace can also be attached to the warp-knitted fabric. For example, shaping elements can be welded on such that the warp-knitted fabric is stretched in one direction when the back brace is in its unstretched original form.

1. A medical aid comprising a mesh-like knitted fabric having an open structure that can be stretched simultaneously in both a longitudinal direction and a transverse direction, wherein the knitted fabric is used in a stretched state in the medical aid, and wherein each of a plurality of sub-elements of the open structure has a width of at least 2.0 mm and at most 10.0 mm in the stretched state.

2. The medical aid according to claim 1, wherein the knitted fabric is a crocheted fabric.

3. The medical aid according to claim 1, wherein the crocheted fabric is a circular-crocheted or flat-crocheted fabric.

4. The medical aid according to any claim 1, wherein the crocheted fabric has weft threads.

5. The medical aid according to claim 1, wherein the knitted fabric is a warp-knitted fabric.

6. The medical aid according to claim 5, wherein the knitted fabric has no pile threads.

7. The medical aid according to claim 1, wherein the knitted fabric is stretched in at least one direction in the medical aid.

8. The medical aid according to claim 1, wherein the medical aid is an orthopedic aid.

9. The medical aid according to claim 1, wherein the medical aid is a bandage or an orthosis.

10. The medical aid according to claim 1, wherein the medical aid is a back brace.

11. The medical aid according to claim 1, wherein the open structure is selected from a group consisting of a mesh structure, a rhombic structure and a honeycomb structure.

12. The medical aid according to claim 5, wherein the knitted fabric contains at least one rubbery-elastic thread and one wrapped thread.

13. The medical aid according to claim 5, wherein a first component of the knitted fabric comprises one double-wrapped rubbery-elastic thread.

14. The medical aid according to any claim 5, wherein the warp-knitted fabric comprises a warp thread having a gauge or thickness of at least 700 dtex to at most 10.5 ktex.

15. The medical aid according to claim 1, wherein at least one shaping element, one support element and/or one zone of inelastic material is attached to the knitted fabric.

16. The medical aid according to claim 15, wherein the at least one shaping element, support element or at least one inelastic element is welded, stitched, attached by hook-and-loop tape, riveted or bonded to the knitted fabric.

17. A method for producing a medical aid having a mesh-like knitted fabric with an open structure that can be stretched simultaneously in both a longitudinal direction and a transverse direction, wherein the knitted fabric is used in a stretched state in the medical aid, and wherein each of a plurality of sub-elements of the open structure has a width of at least 2.0 mm and at most 10.0 mm in the stretched state, the method comprising:
a. prestressing the knitted fabric; and
b. applying at least one shaping element, support element and/or inelastic element to the prestressed knitted fabric.

18. The method according to claim 17, wherein in step b) the at least one shaping element, support element and/or inelastic element is welded, stitched, attached by hook-and-loop tape, riveted or bonded to the prestressed knitted fabric.

19. A bandage comprising a base support, the base support including a mesh-like knitted fabric with an open structure that can be stretched simultaneously in both a longitudinal direction and a transverse direction, wherein the knitted fabric is used in a stretched state in the medical aid, and wherein each of a plurality of sub-elements of the open structure has a width of at least 2.0 mm and at most 10.0 mm in the stretched state.

20. The bandage according to claim 19, wherein the knitted fabric is a compression element of the bandage.
